(19) United States
(12) Patent Application Publication
(10) Pub. No.: US 2013/0210002 A1
(43) Pub. Date: Aug. 15, 2013

(54) METHOD OF ANALYZING CELLULAR CHROMOSOMES

(71) Applicants: Xiuqing Zhang, Guangdong (CN); Zhaoling Xuan, Guangdong (CN); Fang Chen, Guangdong (CN); Fuman Jiang, Guangdong (CN); Jingrong Lin, Guangdong (CN); Peipei Li, Guangdong (CN)

(72) Inventors: Xiuqing Zhang, Guangdong (CN); Zhaoling Xuan, Guangdong (CN); Fang Chen, Guangdong (CN); Fuman Jiang, Guangdong (CN); Jingrong Lin, Guangdong (CN); Peipei Li, Guangdong (CN)

(21) Appl. No.: 13/640,691

(22) PCT Filed: Aug. 13, 2010
(86) PCT No.: PCT/CN2010/001230

§ 371 Date: Apr. 8, 2013

(51) Int. Cl. C12Q 1/68

(52) U.S. Cl. CPC C12Q 1/6869
USPC 435/6.11; 702/20

(57) ABSTRACT

The present invention involves an analysis method of cellular chromosomes, particularly involves a method of analyzing whether a difference exists in the chromosome number between amniotic cells and standard cells by a sequencing method.

### FIELD OF INVENTION

This invention relates to a method of analyzing cellular chromosomes, and particularly relates to a method of analyzing the chromosomes of amniotic cells by sequencing.

### BACKGROUND OF INVENTION

### Fetal Chromosomal Aneuploidy

Fetal chromosomal aneuploidy means a condition that the number of chromosome is not diplontic in a fetal genome. Normally, there are 44 autosomes and 2 sex chromosomes in a human genome in which the male karyotype is (46, XY) and the female karyotype is (46, XX). Fetal chromosomal aneuploidy may refer to the condition of having one more chromosome than a normal diploid fetus, i.e. 47 chromosomes in the fetal genome. Take fetal trisomy 21 for example, compared with a normal diploid fetus, the fetus with trisomy has an extra chromosome 21 with the karyotype of 47, XX (or XY), +21. Also, fetal chromosomal aneuploidy refers to the condition of missing a chromosome in comparison with the normal diploid fetus, i.e. 45 chromosomes in the fetal genome. For example, the fetus with Turner syndrome with the karyotype of 45, XO misses one chromosome, relative to the normal diploid fetus. Fetal chromosomal aneuploidy also refers to the condition that a part of chromosome is lost, or example, translocated trisomy 21 with the karyotype of 45, XX, der (14; 21)(q10; q10), and Cri du chat syndrome with the karyotype of 46, XX (XY),del (5)(p13).

According to incomplete statistics, the birth rate of fetuses with chromosomal aneuploidy is 1/160 in the world, wherein the birth rate of fetal trisomy 21 (T21, Down syndrome) is 1/800, the birth rate of fetal trisomy 18 (T18, Edwards syndrome) is 1/6000, and the birth rate of trisomy 13 (T13, Patau syndrome) is 1/10000. The development of fetuses with other types of the chromosomal aneuploidy stagnates because some developmental stages could not be accomplished, resulting in clinically unreasoned miscarriage at the early stage of gestation (Deborah A. Driscoll, M. D., and Susan Gross, M. D., Prenatal Screening for Aneuploidy[J]. N Engl J Med 2009; 360:2556-62).

### Current Situation of Culturing Amniotic Cells

Amniotic cells are epithelial cells floating in the amniotic fluid, which derive from skin, digestive tracts and respiratory tracts of fetus. The procedure of culturing amniotic cells, enriching the number of fetal nucleated cells, preparing chromosomal specimen, and analyzing the fetal chromosomal karyotype is a golden standard of traditionally clinically diagnosing the chromosomal abnormality of fetuses. This technique is reliable, accurate, and enables the observation of abnormalities of chromosome number and structure. Its disadvantages, however, are that it is time-consuming, it takes 10 days to 3 weeks to yield the result, and culturing failure rate is about 1.00% (THEIN A T, ABDEL-FATTAH S A, KYLE P M, et al., An Assessment of the Ase of Interphase FISH with Chromosome Specific Probes as an Alternative to Cytogenetics in Prenatal Diagnosis [J]. PrenatDiagn, 2000, 20(4): 275-280).

The reason of high failure rate of culturing amniotic cells is that the amniotic cells are aging and pyknotic cells, resulting in harder culturing than that of the other tissues (Changjun Ma, Yuania Chen, Peidan Huo; The Culture of Amniotic Cells and the Method of Preparing the Chromosomal Specimen Thereof [J]. Reproduction and Contraception, 1985, 5 (1): 53-4). Therefore, successful culturing of amniotic cells plays a critical role in the process of detecting chromosomal aneuploidy. Because of the high requirement of culturing amniotic cells, relatively few viable cells in the amniotic fluid of some of pregnant women, relatively few harvested cells with division phases, and poor chromosomal shape, it is difficult to count and analyze a sufficient number of cells, and detect chromosomes. Furthermore, amniocentesis is a risk with about 2-3% of pregnant women suffering complications, such as uterine contractions, abdominal swelling, tenderness, vaginal bleeding, infection, water leakage, or fetal injury. It would be unacceptable for a pregnant woman if being asked to do second amniocentesis if the culturing of amniotic cells fails or the harvested cells are not sufficient to count and analyze. Moreover, the amniocentesis is generally performed at 16-20 weeks' gestation, once the culture fails, many pregnant women's gestational period has advanced too far, and thus have to undergo cordocentesis with even higher risk. After the culturing of amniotic cells, the analysis of karyotype requires a lot of labor and costs such that many hospitals cannot afford the procedure, causing great difficulties in the clinical spreading and application of amniocentesis.

With the continuous development of sequencing techniques, it is being increasingly applied in the detection and analysis of the chromosome number. Dennis Lo et al. used the peripheral blood of a pregnant woman as experimental material to examine the abnormality of chromosome number by means of massive sequencing based on mathematical statistics methods (Y. M. Dennis Lo, et al., Quantitative Analysis of Fetal DNA in Maternal Plasma and Serum: Implications for Noninvasive Prenatal Diagnosis. Am. J. Hum. Genet. 62:768-775, 1998). But this method cannot completely replace amniocentesis in clinical application, because of some defects occurring in the technique: the cell-free DNAs in plasma are fragmented DNAs, they cannot form a complete genome on their own, the aneuploidy, translocation or mosaicism of the chromosomes other than chromosomes 21, 18, and 13 fails to be detected or have a low detective accuracy.

### SUMMARY OF INVENTION

In order to overcome the missed results of detection caused by the sequencing of peripheral blood, and resolve the high failure rate of the culturing of amniotic cells, this invention, in combination with the advantages of the analysis of karyotype by amniocentesis and the method of sequencing the cell-free DNAs in plasma, utilizes a method of detecting chromosomal aneuploidy based on massive sequencing of amniotic cells, including the steps of drawing amniotic cells, isolating DNA, conducting high-throughout sequencing, analyzing the obtained data, and acquiring detection results.

In one aspect of the invention, a method of using high-throughout sequencing technique to analyze the chromosomal information of a subject's cells is provided, comprising the steps of:

a. randomly breaking the genomic DNA of the cells, obtaining DNA fragments with a certain size, and sequencing them;

b. strictly aligning the DNA sequences sequenced in step a to the reference sequence of the human genome to obtain the information about the DNA sequences located on a particular chromosome;

c. for the particular chromosome N, determining the total number of the sequences mapped to a sole region of the chromosome, among the above-sequenced DNA sequences, thereby making ChrN % for chromosome N, i.e. ratio of the total number (S1) of the sequences mapped to the sole place of chromosome N, among the above-sequenced DNA sequences, to the total number (S2) of the sequences located on all chromosomes, among the above-sequenced DNA sequences: ChrN %=S1/S2;

d. comparing ChrN % for chromosome N with ChrN % for the corresponding chromosome coming from standard cells to determine whether there exists a difference between the chromosome of the cells and the corresponding one of the standard cells.

In the invention, the cells may be, for example, amniotic cells, wherein the amniotic cells may be uncultured amniotic cells or cultured amniotic cells. In one embodiment of the invention, to avoid culturing amniotic cells, the amniotic cells are uncultured amniotic cells.

In the invention, the genomic DNA of the cells may be obtained by traditional methods of isolating DNA, such as salting-out, column chromatography, and SDS, preferably by column chromatography. The so-called column chromatography involves using cell lysis buffer and protease K to treat amniotic cells or tissues to expose naked DNA molecules, making them pass through a silica membrane column capable of binding negatively charged DNA molecules, to which the genomic DNA molecules in the system are reversibly adsorbed, removing the impurities such as proteins or lipids by washing buffers, and diluting by purifying buffers to obtain the DNA of amniotic cells (for more details about specific principles and methods, see the product manuals for product No. 56304 from Qiagen and product DP316 from Tiangen).

In the invention, the DNA molecules are randomly broken by restriction cleavage, atomization, ultrasound, or HydroShear method. HydroShear method is preferably used (when the solution containing DNA is flowing through a passage with small section, the flowing rate is accelerated, creating a force enough to destruct suddenly the DNA to produce DNA fragments in various sizes depending on the flowing rate and the section area. For more details about specific principles and methods, see product manuals of HydroShear from Life Sciences Wild). In this way the DNA molecules are broken into fragments with a narrow range of sizes, of which major bands generally range from 200 bp to 300 bp in size.

The sequencing method adopted in the invention may be the second generation sequencing method such as Illumina/Solexa or ABI/SOLiD. In one embodiment of the invention, the sequencing method is Illumina/Solexa and the resultant sequences are fragments with 35 bp in size.

When the DNA molecules to be examined are from multiple samples, each sample may be attached a different tagged sequence index so as to be processed during the process of sequencing (Micah Hamady, Jeffrey J Walker, J Kirk Harris et al., Error-correcting Barcoded Primers for Pyrosequencing Hundreds of Samples in Multiplex. Nature Methods, 2008, March, Vol. 5 No. 3).

In the invention, the reference sequence of the human genome is produced after the shield of the repeated sequences within the human genome sequence, for example, the latest version of the reference sequence of the human genome in the NCBI database. In one embodiment of the invention, the human genome sequence is the reference sequence of the human genome as shown in version 36 (NCBI Build 36) of NCBI database.

In the invention, aligning strictly with the reference sequence of the human genome means that the adopted method of alignment is a fault-intolerant alignment of the sole region located in the reference sequence of the human genome. In one embodiment of the invention, alignment software Eland (a software package provided by Illumina) was used, and the method adopted was an absolute, fault-intolerance alignment.

In the invention, when the said DNA sequences is a sequence which is able to be located at a sole region of the reference sequence of the human genome, it is defined as sole sequence represented by Unique reads. In the invention, for the purpose of avoiding the interference of the repeated sequences, it is needed to remove those DNA sequences located at the regions of tandem repeats and transpositional repeats within the reference sequence of the human genome and merely take into account those DNA sequences, i.e. sole sequences, which may be located at a sole region. Generally, of all the sequenced DNA sequences, about a quarter to a third of DNA sequence are able to be located at a sole region of the genome, i.e. sole sequences. The statistical number of these sole sequences represents the distribution of the DNA sequences on the genomic chromosomes.

Therefore, the sole sequences can assist in the localization of each DNA sequence that is produced by breaking and sequencing the DNA molecules isolated from amniotic cells on a particular chromosome. ChrN % is values produced by normalizing the sole sequences found on different chromosomes, and the values are merely relevant to the size of a particular chromosome rather than the amount of the data being sequenced. Thus the values can be used to analyze the information on individual 46 chromosomes. Therefore, ChrN % is basic value to conduct a chromosomal analysis.

In the invention, whether there exists a difference between the number of a particular chromosome in the cellular samples and the standard cells can be determined by drawing a boxplot, wherein a sample for which ChrN % corresponds to an outlier that goes beyond 1.5-3 time or above 3 times the interquartile range is determined to differ from the standard cells in the chromosome number, i.e. aneuploidy.

In the invention, determining whether there exists a difference between a particular chromosome respectively in the said cellular samples and in the standard cellular samples may be accomplished by using “z score_ChrN” to indicate the deviation of ChrN % for the said cellular samples from ChrN % for the standard cellular samples.

Specifically, z score_ChrN=(ChrN % for a particular chromosome from detection samples−ChrN % mean (mean_ChrN %) for the particular chromosome)/ChrN % standard deviation (S.D._ChrN %).

If z score_ChrN is extremely large or small, it means that the deviation of the chromosome number in the cellular detection sample from that of the normal sample is significant. When it reaches a given level of significance, it may be believed that there is an apparent difference between the former number and the latter number.

In the invention, the average value of ChrN % for a particular chromosome from the standard cellular samples may be determined according to ChrN % for the chromosome from such as at least 10, 20, 30, 50, or 100 standard cellular samples.

In the invention, the standard cellular samples are the samples of human cells in which the number of the chromosomes is diploid. A normal male cell has 44 autosomes and 2 different sex chromosomes, (46, XY). On the other hand, a normal female cell has 44 autosomes and 2 identical sex chromosomes, (46, XX).

In the invention, the ChrN % standard deviation (S.D._ChrN %) for a particular chromosome from the standard cellular samples may be determined according to the ChrN % for the chromosomes, such as at least 10, 20, 30, 50, or 100 standard cellular samples.

In one embodiment of the invention, the standard cellular samples have 20 samples from normal males and 10 samples from normal females, numbered, respectively, with 1, 2, . . . , 30, in which Nos. 1-20 are the detection samples from normal males, Nos. 21-30 are the detection samples from normal females. The average value of ChrN % (mean_ChrN %) for the standard cellular samples is calculated as follows:

$$Mean\_ChrN \% = \frac{1}{30} {\sum }_{m = 1}^{30} ChrC\_M )$$

$$( wherein N represents autosomes 1 \text{-} 22 , \\ M represents normal samples Nos . 1 \text{-} 30 )$$

$$Mean\_ChrX \% ( male ) = \frac{1}{20} {\sum }_{m = 1}^{20} ChrX\_M$$

$$( M represents normal male samples Nos . 1 \text{-} 20 )$$

$$Mean\_ChrY \% ( male ) = \frac{1}{20} {\sum }_{m = 1}^{20} ChrY\_M$$

$$( M represents normal male samples Nos . 1 \text{-} 20 )$$

$$Mean\_ChrX \% ( female ) = \frac{1}{10} {\sum }_{m = 21}^{30} ChrX\_M$$

$$( M represents normal female samples Nos . 21 \text{-} 30 )$$

$$Mean\_ChrY \% ( female ) = \frac{1}{10} {\sum }_{m = 21}^{30} ChrY\_M$$

$$( M represents normal female samples Nos . 21 \text{-} 30 )$$

(Note: due to the fluctuation of sequencing and a large number of gaps existing on Y chromosome of the reference sequence, it results in that even for the normal female samples there are a few DNA sequences aligned with Y chromosome. As compared with males, however, the ChrN % for females is much less than that for males. In the examples, the ChrN % for females is around 0.004, whereas the ChrN % for males is around 0.114.)

Based on each ChrN % mean (mean_ChrN %) for the standard cellular samples obtained by the method described above, the ChrN % standard deviation (S.D._ChrN %) is calculated with the following formula:

$$S . D . \_ChrN \% = \frac{1}{30} {\sum }_{m = 1}^{30} {( ChrN\_M - mean\_ChrN )}^{2}$$

$$( wherin N represents autosomes 1 \text{-} 22 )$$

$$S . D . \_ChrX \% ( male ) = \frac{1}{20} {\sum }_{m = 1}^{20} {( ChrX\_M - mean\_ChrX )}^{2}$$

$$( M represents normal male samples Nos . 1 \text{-} 20 )$$

$$S . D . \_ChrY \% ( male ) = \frac{1}{20} {\sum }_{m = 1}^{20} {( ChrX\_M - mean\_ChrX )}^{2}$$

$$( M represents normal male samples Nos . 1 \text{-} 20 )$$

$$S . D . \_ChrX \% ( female ) = \frac{1}{20} {\sum }_{m = 1}^{20} {( ChrX\_M - mean\_ChrX )}^{2}$$

$$( M represents normal female samples Nos . 21 \text{-} 30 )$$

$$S . D . \_ChrY \% ( female ) = \frac{1}{20} {\sum }_{m = 1}^{20} {( ChrX\_M - mean\_ChrX )}^{2}$$

$$( M represents normal female samples Nos . 21 \text{-} 30 )$$

Since there is a missed X chromosome replaced by Y chromosome among male chromosomes in contrast to female chromosomes, and the whole length of the X chromosome is about 155M, whereas the Y chromosome is about 59M. In detecting these sex chromosomes, it is necessary to establish a set of normal distribution curves concerning ChrX % or ChrY % for different agendas. The most accurate analysis for X chromosome can be obtained from the different agenda-based normal distribution curves.

In one embodiment of the invention, 30 standard cellular samples were selected to conduct the chromosomal analysis. Then a normal distribution curve was established under the requirement of significance level (such as 0.1%) for normal distribution reached in the instance of having a difference between the number of simulated chromosomes and that in standard cells. Thus, the instance of the absolute value of the z score_ChrN being determined to be below 3 was defined by the number of chromosomes being the same as that in the standard cells. On the basis of the results above, then the chromosomes of the detection samples were analyzed as follows:

If the absolute value of the z score amounts to 3, then the samples have a 99.9% probability that they are not among the normally distributed population, i.e. outliers. This means that the chromosome number of the detected cells differs from that of the standard cells, i.e. chromosomal aneuploidy.

If the absolute value of the z score is less than 3, then the samples have a 99.9% probability that they are normal samples, which means that the chromosome number of the detected cells is the same as that of the standard cells.

If the absolute value of the z score is greater than 3, then the samples have a 99.9% probability that they are abnormal samples, which means that the chromosome number of the detected cells differs from that of the standard cells, i.e. chromosomal aneuploidy.

Further, in the invention, if the absolute value of the z score is greater than 3, for the specific instance of chromosomal aneuploidy occurring in the detected cells, the Z reference value (cutoff value) may be used to determine it. The Z reference value is calculated with the following formula:

Z=(mean_ChrN %×0.5×X%)/S.D._ChrN %

When N represents autosomes, mean_ChrN % and S.D._ChrN % are the means for all of the samples of the standard cells. When N represents sex chromosomes, mean_ChrN % and S.D._ChrN % are the means for the samples of the standard cells of respective agenda;

X may be any integer between, inclusive, −100 and 100, such as −100, −90, −80, −70, −60, −50, −40, −30, −20, −10, 0, 10, 20, 30, 40, 50, 60, 70, 80, 90, 100.

In one embodiment of the invention, when X amounts to 100, it represents that the cellular detection samples have one more chromosome than the standard cells. In one embodiment of the invention, when X amounts to −100, it represents that the cellular detection samples have one less chromosome than the standard cells. When X amounts to 50, it represents that the cellular detection samples have extra half of one chromosome than the standard cells. In one embodiment of the invention, when X amounts to 50, it represents that the cellular detection samples lacks half of one chromosome in comparison with the standard cells.

In the invention, in calculating the Z reference value for sex chromosomes, mean_ChrN % and S.D._ChrN % are calculated for either female samples or the male samples. That is:

For the male samples, the Z reference value reached may be (mean_ChrN % (male)×0.5×X %)/S.D._ChrN % (male);

For the female samples, Z reference value reached may be (mean_ChrN % (female)×0.5×X %)/S.D._ChrN % (female).

When the absolute value of the z score_ChrN is greater than or equal to 3 and reaches the absolute value of the Z reference value, there is a significant difference between the number of a particular chromosome in the detected cells and that in the standard cells equal to X %. For example, when X amounts to 50, it represents that the detected cells have extra half of the particular chromosome compared with the standard cells; when X amounts to 100, it represents that the detected cells have one particular chromosome more than the standard cells.

In one embodiment of the invention, the specific method of analyzing the chromosomes of amniotic cells includes the following steps:

### 1. DNA Isolation and Sequencing

A library was built according to the modified Illumina/Solexa standard procedure of building a library after the DNA of amniotic cells was isolated in accordance with the manual from Tiangen Micro Kit. Then adapters for sequencing were added to the both ends of the randomly broken DNA fragments. During the process, a different tagged sequence (index) was attached to each of the samples such that multiple samples could be differentiated in the data obtained from one-time sequencing.

### 2. Alignment and Statistic

After sequencing by using the second generation sequencing technique, known as Illumina/Solexa sequencing (other alternate sequencing methods can also be used to achieve the same or similar effects), the fragmented DNA sequences of a specified size were produced for each sample, which were subjected to alignment strictly with the reference sequence of the human genome. Thus, the information was obtained on the location of the sequences at the corresponding regions of the genome.

Such a restricted alignment was required because it could not be determined from which chromosome a given DNA sequence originated if fault-tolerant alignment or the alignment with multiple regions was allowed. This would be unfavorable for the subsequent analysis of the data.

The total number of the sole sequences located on each chromosome was calculated by taking each chromosome as a unit, thereby making the ChrN % for each chromosome, i.e. ratio of the total number (S1) of the sequences among the above-sequenced DNA sequences, which are located at the sole place of chromosome N, to the total number (S2) of the sequences among the above-sequenced DNA sequences, which are located on all chromosomes: ChrN %=S1/S2.

This is a method of normalization for different samples having a different sequencing amount. Because amniotic cells contain the whole information of 46 chromosomes, theoretically the total number of the sequences located on a given chromosome is directly proportional to the length of the chromosome.

For example, chromosome 1 is the largest chromosome (about 247 M) in the human genome, whereas chromosome 21 is the smallest chromosome (about 47 M) in the human genome, therefore, given a certain total amount of sequencing, the sequencing results from normal diploid amniotic cells are nearly a fixed value. Although in some sequencing and experimental conditions, the ChrN % is not directly proportional to the size of chromosomes, it is usually a fixed value.

### 3. Analysis of the Data

By a boxplot boxplotanalysis, it may be directly determined whether the cellular detection samples are likely to differ from the standard cells in the chromosome number. The samples with abnormal values are directly considered as suspect samples, and the others are considered as standard cellular samples. The detailed process is as follows:

In the invention, in order to help with the data analysis, a boxplot (used for differentiate abnormal values in mathematical statistics) involving the ChrN % produced above is adopted to determine suspect samples. The specific drawing process is as follows:

1) calculating the upper-quartile (75%), median (50%), and lower-quartile (25%);

2) calculating the difference, interquartile range (IQR), between the upper-quartile and lower-quartile;

3) drawing the upper and lower ranges of a boxplot with the upper limit being the upper-quartile and the lower limit being the lower-quartile, and drawing a horizontal line where the median lies inside the box;

4) values which are 1.5 times greater than the upper-quartile of the interquartile range or 1.5 times less than the lower-quartile of the interquartile range are classified as outliers.

5) beyond the outliers, drawing a horizontal line across the two value points closest to the upper margin and the lower margin, respectively, as a “whisker” of the boxplot.

6) extreme outliers going beyond a distance three times longer than the interquartile range are represented with star points; milder outliers that lie within 1.5-3 times as the distance of the interquartile are represented with hollow points.

The bold line in the middle of the box represents median values, and the upper and lower boarders represent the upper and lower quartiles, respectively. Outliers are defined by the points deviating from 1.5 times the distance between the upper quartile and lower quartile. For example, when the detection samples are standard cellular samples, the ChrN % corresponding to their chromosomes is a fixed value (for example, 1). When the ChrN % corresponding to their chromosomes is 1.5, then the difference can be considered greatly significant, thereby making the samples suspected samples. That is, it is likely that they are samples differed from the standard cells in the chromosome number.

If needed, the ChrN % mean and standard deviation (S.D._ChrN %) may be determined, respectively, by the ChrN % for a particular chromosome corresponding to the standard cellular samples. Then the z score_ChrN for the chromosome from the suspected samples are calculated with the following formula:

z score_ChrN=(ChrN % for the particular chromosome from the suspected samples−ChrN % mean)/S.D._ChrN %

If the absolute value of the z score_ChrN is greater than or equal to 3, there is a difference between the number of a particular chromosome in the cellular samples and that in the standard cells.

Further, in the invention, for the specific instance of an abnormal chromosome number occurring in the cells, reference to the Z reference value (cutoff value) may be used to determine it. Value Z is calculated with the following formula:

Z=(mean_ChrN %×0.5×X %)/S.D._ChrN %

When N represents autosomes, the mean_ChrN % and S.D._ChrN % are the mean of all the samples of the standard cells. When N represents sex chromosomes, the mean_ChrN % and S.D._ChrN % are the mean of the samples of the standard cells of the respective agenda;

X is assigned to be 50 or 100. Correspondingly, when X is 100, it represents that the cellular detection samples have one more chromosome than the standard cells. When X is 50, it represents that the cellular detection samples have extra half of one chromosome than the standard cells.

In the invention, in calculating the Z reference value for sex chromosomes, the mean_ChrN % and S.D._ChrN % are the mean for either female samples and male samples, that is:

For the male samples, the Z reference value reached is (mean_ChrN % (male)×0.5×X %)/S.D._ChrN % (male);

For the female samples, the Z reference value reached is (mean_ChrN % (female)×0.5×X %)/S.D._ChrN % (female).

When the absolute value of the z score_ChrN is greater than or equal to 3 and reaches the absolute value of the Z reference value, there is a X % difference between the number of a particular chromosome in the cells and that in the standard cells.

### ADVANTAGES OF THE INVENTION

The invention can be used for the analysis of cells, such as amniotic cells. In the invention, DNA can directly be isolated from amniotic cells to be detected without a subculture, which greatly decreases the difficulties such as uneasy attachment, insufficient number, or failure of culture caused by the culture of amniotic cells.

By using the characteristic of amniotic cells containing the entire genomic information about a fetus, the invention is able to make an analysis of the aneuploidy of all of the chromosomes of the cells, rather than examine only the sex chromosomes X and Y and chromosomes 21, 18, 13.

Besides, though the method of determination involved in the invention, as compared with plasma samples, is also dependent on approximately normal distribution established on standard cellular samples, such dependency on the standard cellular samples is greatly reduced. Additionally, abnormal samples can be directly determined from data abnormalities, assuming sufficient data.

By using the method of the invention, a large number of cellular detection samples can be subjected to batch analysis. Hundreds of thousands of cellular detection samples can be detected at one time, thereby greatly saving labors and costs.

### BRIEF DESCRIPTION OF FIGURES

FIG. 1 shows a boxplot depicted in accordance with 53 cellular detection samples, in which the abscissa represents the chromosome number, and the ordinate represents the ChrN % value.

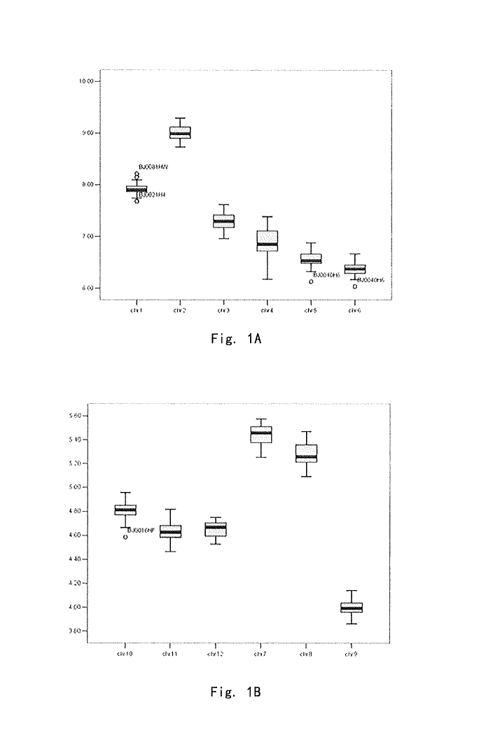

FIG. 1A shows chromosomes 1-6, FIG. 1B shows chromosomes 7-12, FIG. 1C shows chromosomes 13-17, and FIG. 1D shows chromosomes 18-22.

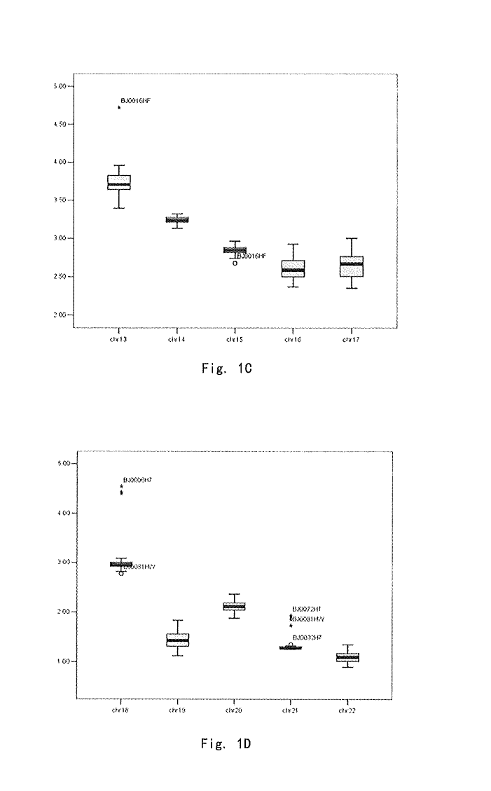

### DETAILED DESCRIPTION OF THE PREFERRED EMBODIMENTS

The embodiment of the invention will be described in detail in combination with the following examples. A person skilled in the art would appreciate, however, that the following examples are merely intended to make a description of the invention and would not be regarded as the limitation of the scope of the invention. If specific conditions are not specified in the examples, these examples are performed in accordance with commonly used conditions or those advised by manufacturers. If the sources of the reagents or equipment or instruments used in the invention are not specified, such as their manufacturers, all of them are commercially available. The used linkers for sequencing and the tagged sequences index come from Multiplexing Sample Preparation Oligonutide Kit provided by Illumina.

In the following parentheses is manufacturers' product number for each of the reagents or kits.

### Example 1

### Chromosomal Analysis of Uncultured Amniotic Cell

### 1. Isolation and Sequencing of DNA

DNA of amniotic cells was isolated according to the procedure of manipulation of a small amount of genome of Tiangen Micro Kit (DP316), and quantitated with Qubit (Invitrogen, the Quant-iT™ dsDNA HS Assay Kit). The total amount of the isolated DNA varied from 100 ng to 500 ng.

The isolated DNA was either the entire genomic DNA or partially degraded smear-like DNA. A DNA library was built under the standard library-building procedure provided by the modified Illumina/Solexa. Adapters were added to both ends of randomly broken DNA molecules, and attached with different tagged sequence indexes. Then these molecules were hybridized with complementary adapters on the surface of a flow cell, and allowed to be clustered in particular conditions. 36 sequencing cycles were run on an Illumina Genome Analyzer II, producing DNA fragments with 35 bp.

Specifically, Diagenode Bioruptor was used to randomly break about 100-500 ng of DNA isolated from amniotic cells into 300 bp fragments. 100-500 ng of initially broken DNA was used to build a library under Illumina/Solexa. See the prior art for a detailed procedure (Illumina/Solexa manual for standard library-building provided by Illumina's website). The size of the DNA library was determined by way of 2100 Bioanalyzer (Agilent), and the inserted fragments were 300 bp. After accurate quantitation by QPCR, sequencing was performed.

In the example, batch sequencing was conducted of 53 DNA samples isolated from amniotic cells according to Cluster Station and GA II×(SE sequencing) officially published by Illumina/Solexa.

### 2. Alignment and Statistics

Refer to the prior art (see the manual concerning Pipeline method provided at Illumina's website), the sequence information obtained in step 1 was subjected to one single Pipeline process, and sequences with low quality were removed, finally resulting in ELAND alignment result against the reference sequence of the human genome of NCBI version 36. Then the number of the sole sequences located on chromosomes was statistically analyzed.

The ChrN % for 22 chromosomes and the X/Y chromosome respectively from 53 samples was calculated and a boxplot (see FIG. 2) was drawn based on the data. The ChrN % for a particular chromosome N in particular sample M is calculated with the following formula:

Percentage of a particular chromosome in detection sample M, ChrN %=the total number of the sole sequences contained in sample M and located on the corresponding chromosome of the reference sequence through alignment (S1)/the total number of the sole sequences contained in sample M and located on all of the chromosomes of the reference sequence through alignment (S2).

### 3. Data Analysis

According to the boxplot drawn in step 2, it was firstly determined whether an outlier existed. That is, as compared with the upper and lower boarders, if a suspected sample deviated far from the point that was 1.5 times the difference between the upper-quartile and the lower-quartile away, it was likely that it differed from the standard samples in chromosome number.

Specifically, the distribution of the boxplot was observed, and 8 suspected samples (sample Nos. P1-P8) were detected. A normal distribution was established by using as standard samples the data concerning 20 normal males and 10 normal females, chosen randomly from the remaining 45 standard cellular samples after the suspected samples were removed. The ChrN % mean (mean_ChrN %) for each chromosome is designated by mean_ChrN % and standard deviation (S.D._ChrN %) is given in table 1.

TABLE 1
ChrN %, mean, and standard deviation (S.D.) for each chromosome in standard cells

| | Chr1 | Chr2 | Chr3 | Chr4 | Chr5 | Chr6 | Chr7 | Chr8 | Chr9 | Chr10 | Chr11 | Chr12 |
|---|---|---|---|---|---|---|---|---|---|---|---|---|
| Number | % | % | % | % | % | % | % | % | % | % | % | % |
| 1 | 7.850 | 9.295 | 7.542 | 7.381 | 6.876 | 6.660 | 5.499 | 5.468 | 3.942 | 4.747 | 4.603 | 4.744 |
| 2 | 7.965 | 9.088 | 7.483 | 6.907 | 6.686 | 6.482 | 5.494 | 5.331 | 4.040 | 4.834 | 4.645 | 4.699 |
| 3 | 7.935 | 9.121 | 7.414 | 6.989 | 6.649 | 6.491 | 5.500 | 5.324 | 4.035 | 4.834 | 4.704 | 4.695 |
| 4 | 7.866 | 9.237 | 7.618 | 7.353 | 6.875 | 6.585 | 5.530 | 5.424 | 3.976 | 4.722 | 4.546 | 4.743 |
| 5 | 7.847 | 9.179 | 7.371 | 7.100 | 6.784 | 6.509 | 5.535 | 5.374 | 3.988 | 4.843 | 4.587 | 4.721 |
| 6 | 7.752 | 9.247 | 7.617 | 7.337 | 6.871 | 6.600 | 5.573 | 5.401 | 3.960 | 4.776 | 4.564 | 4.742 |
| 7 | 7.920 | 9.149 | 7.501 | 7.178 | 6.826 | 6.607 | 5.515 | 5.360 | 4.003 | 4.792 | 4.598 | 4.748 |
| 8 | 8.089 | 8.953 | 7.289 | 6.614 | 6.532 | 6.317 | 5.462 | 5.237 | 4.059 | 4.922 | 4.711 | 4.698 |
| 9 | 8.155 | 9.005 | 7.190 | 6.459 | 6.565 | 6.267 | 5.529 | 5.238 | 4.154 | 4.950 | 4.819 | 4.573 |
| 10 | 7.961 | 9.133 | 7.362 | 6.768 | 6.627 | 6.418 | 5.520 | 5.280 | 3.987 | 4.822 | 4.674 | 4.734 |
| 11 | 8.079 | 8.980 | 7.217 | 6.602 | 6.493 | 6.294 | 5.504 | 5.219 | 4.078 | 4.921 | 4.770 | 4.712 |
| 12 | 7.953 | 9.205 | 7.499 | 7.173 | 6.786 | 6.533 | 5.535 | 5.343 | 3.977 | 4.789 | 4.654 | 4.664 |
| 13 | 7.986 | 9.051 | 7.360 | 6.848 | 6.701 | 6.446 | 5.541 | 5.376 | 4.065 | 4.822 | 4.701 | 4.689 |
| 14 | 8.111 | 9.040 | 7.210 | 6.905 | 6.548 | 6.364 | 5.472 | 5.341 | 4.012 | 4.884 | 4.725 | 4.677 |
| 15 | 8.032 | 9.002 | 7.325 | 6.818 | 6.571 | 6.369 | 5.537 | 5.345 | 4.064 | 4.913 | 4.672 | 4.682 |
| 16 | 8.075 | 8.977 | 7.199 | 6.664 | 6.515 | 6.349 | 5.480 | 5.311 | 4.068 | 4.856 | 4.741 | 4.703 |
| 17 | 7.878 | 9.184 | 7.502 | 7.221 | 6.793 | 6.592 | 5.523 | 5.405 | 3.990 | 4.763 | 4.588 | 4.727 |
| 18 | 7.873 | 9.165 | 7.502 | 7.194 | 6.775 | 6.553 | 5.496 | 5.384 | 4.025 | 4.786 | 4.629 | 4.755 |
| 19 | 7.911 | 9.119 | 7.574 | 7.286 | 6.830 | 6.507 | 5.539 | 5.365 | 3.949 | 4.781 | 4.577 | 4.695 |
| 20 | 8.013 | 9.186 | 7.394 | 6.822 | 6.634 | 6.384 | 5.475 | 5.297 | 4.033 | 4.849 | 4.657 | 4.678 |
| 21 | 7.739 | 8.991 | 7.232 | 6.822 | 6.503 | 6.260 | 5.374 | 5.214 | 3.941 | 4.797 | 4.629 | 4.591 |
| 22 | 7.887 | 8.962 | 7.178 | 6.730 | 6.471 | 6.259 | 5.353 | 5.215 | 3.978 | 4.754 | 4.608 | 4.566 |
| 23 | 7.921 | 8.903 | 7.258 | 6.803 | 6.438 | 6.285 | 5.372 | 5.234 | 3.973 | 4.739 | 4.624 | 4.626 |
| 24 | 7.807 | 8.900 | 7.271 | 6.954 | 6.526 | 6.320 | 5.357 | 5.283 | 3.968 | 4.772 | 4.585 | 4.609 |
| 25 | 7.681 | 9.020 | 7.359 | 7.172 | 6.685 | 6.440 | 5.421 | 5.255 | 3.922 | 4.667 | 4.502 | 4.665 |
| 26 | 7.892 | 8.827 | 7.205 | 6.728 | 6.529 | 6.305 | 5.363 | 5.123 | 3.914 | 4.847 | 4.616 | 4.557 |
| 27 | 8.071 | 8.730 | 6.993 | 6.186 | 6.159 | 6.034 | 5.267 | 5.150 | 4.044 | 4.919 | 4.790 | 4.556 |
| 28 | 7.878 | 8.771 | 7.059 | 6.452 | 6.418 | 6.210 | 5.318 | 5.208 | 3.962 | 4.912 | 4.593 | 4.613 |
| 29 | 7.803 | 8.985 | 7.335 | 6.934 | 6.613 | 6.382 | 5.428 | 5.268 | 3.892 | 4.744 | 4.510 | 4.658 |
| 30 | 7.992 | 8.818 | 7.126 | 6.540 | 6.363 | 6.167 | 5.329 | 5.089 | 4.028 | 4.820 | 4.689 | 4.548 |
| mean | 7.931 | 9.041 | 7.339 | 6.898 | 6.621 | 6.400 | 5.461 | 5.295 | 4.001 | 4.819 | 4.644 | 4.669 |
| S.D. | 0.116 | 0.146 | 0.163 | 0.300 | 0.172 | 0.148 | 0.082 | 0.091 | 0.057 | 0.069 | 0.078 | 0.065 |
| | Chr13 | Chr14 | Chr15 | Chr16 | Chr17 | Chr18 | Chr19 | Chr20 | Chr21 | Chr22 | ChrX | ChrY |
| number | % | % | % | % | % | % | % | % | % | % | % | % |
| 1 | 3.917 | 3.294 | 2.762 | 2.401 | 2.380 | 3.051 | 1.120 | 1.966 | 1.295 | 0.920 | 2.163 | 0.126 |
| 2 | 3.743 | 3.279 | 2.885 | 2.595 | 2.650 | 2.980 | 1.431 | 2.148 | 1.296 | 1.088 | 2.128 | 0.123 |
| 3 | 3.770 | 3.268 | 2.914 | 2.565 | 2.602 | 3.027 | 1.434 | 2.112 | 1.270 | 1.073 | 2.154 | 0.119 |
| 4 | 3.944 | 3.254 | 2.801 | 2.416 | 2.402 | 3.080 | 1.140 | 2.000 | 1.279 | 0.914 | 2.170 | 0.126 |
| 5 | 3.880 | 3.234 | 2.866 | 2.545 | 2.576 | 3.049 | 1.354 | 2.064 | 1.275 | 1.050 | 2.154 | 0.112 |
| 6 | 3.909 | 3.322 | 2.792 | 2.393 | 2.389 | 3.089 | 1.139 | 1.966 | 1.309 | 0.910 | 2.217 | 0.127 |
| 7 | 3.868 | 3.305 | 2.833 | 2.451 | 2.481 | 3.002 | 1.266 | 2.033 | 1.327 | 0.942 | 2.165 | 0.129 |
| 8 | 3.585 | 3.258 | 2.943 | 2.800 | 2.863 | 2.996 | 1.685 | 2.262 | 1.296 | 1.223 | 2.091 | 0.116 |
| 9 | 3.590 | 3.205 | 2.926 | 2.860 | 2.960 | 2.879 | 1.669 | 2.271 | 1.339 | 1.240 | 2.046 | 0.111 |
| 10 | 3.714 | 3.305 | 2.873 | 2.677 | 2.767 | 3.001 | 1.539 | 2.168 | 1.307 | 1.154 | 2.085 | 0.122 |
| 11 | 3.638 | 3.303 | 2.927 | 2.787 | 2.872 | 2.947 | 1.687 | 2.244 | 1.294 | 1.275 | 2.041 | 0.115 |
| 12 | 3.823 | 3.285 | 2.829 | 2.493 | 2.504 | 3.012 | 1.323 | 2.068 | 1.293 | 1.025 | 2.116 | 0.120 |
| 13 | 3.734 | 3.282 | 2.819 | 2.676 | 2.670 | 3.002 | 1.479 | 2.100 | 1.299 | 1.097 | 2.135 | 0.121 |
| 14 | 3.647 | 3.274 | 2.894 | 2.746 | 2.758 | 2.939 | 1.501 | 2.256 | 1.308 | 1.147 | 2.120 | 0.121 |
| 15 | 3.720 | 3.262 | 2.891 | 2.663 | 2.730 | 2.976 | 1.551 | 2.176 | 1.287 | 1.164 | 2.122 | 0.130 |
| 16 | 3.584 | 3.269 | 2.970 | 2.767 | 2.868 | 2.965 | 1.667 | 2.186 | 1.270 | 1.277 | 2.125 | 0.114 |
| 17 | 3.864 | 3.246 | 2.827 | 2.475 | 2.476 | 3.036 | 1.228 | 2.024 | 1.297 | 0.963 | 2.277 | 0.120 |
| 18 | 3.853 | 3.315 | 2.844 | 2.506 | 2.489 | 3.040 | 1.229 | 2.071 | 1.299 | 0.963 | 2.129 | 0.124 |
| 19 | 3.950 | 3.312 | 2.845 | 2.457 | 2.484 | 3.065 | 1.234 | 2.031 | 1.265 | 0.962 | 2.136 | 0.125 |
| 20 | 3.731 | 3.285 | 2.886 | 2.645 | 2.720 | 2.906 | 1.485 | 2.155 | 1.299 | 1.151 | 2.200 | 0.114 |
| 21 | 3.690 | 3.224 | 2.829 | 2.522 | 2.618 | 2.919 | 1.384 | 2.119 | 1.269 | 1.107 | 4.224 | 0.003 |
| 22 | 3.642 | 3.223 | 2.877 | 2.578 | 2.673 | 2.910 | 1.422 | 2.137 | 1.259 | 1.087 | 4.226 | 0.004 |
| 23 | 3.680 | 3.234 | 2.824 | 2.536 | 2.585 | 2.968 | 1.380 | 2.098 | 1.265 | 1.062 | 4.187 | 0.004 |
| 24 | 3.707 | 3.223 | 2.809 | 2.501 | 2.580 | 2.933 | 1.372 | 2.039 | 1.262 | 1.042 | 4.177 | 0.004 |
| 25 | 3.829 | 3.214 | 2.740 | 2.388 | 2.390 | 2.978 | 1.211 | 1.975 | 1.259 | 0.929 | 4.293 | 0.004 |
| 26 | 3.679 | 3.232 | 2.859 | 2.579 | 2.707 | 2.918 | 1.485 | 2.081 | 1.262 | 1.129 | 4.159 | 0.003 |
| 27 | 3.404 | 3.207 | 2.912 | 2.899 | 2.968 | 2.893 | 1.802 | 2.344 | 1.263 | 1.330 | 4.077 | 0.003 |
| 28 | 3.525 | 3.190 | 2.869 | 2.771 | 2.852 | 2.903 | 1.647 | 2.215 | 1.278 | 1.227 | 4.127 | 0.003 |
| 29 | 3.786 | 3.177 | 2.778 | 2.456 | 2.498 | 2.980 | 1.258 | 2.037 | 1.271 | 0.986 | 4.216 | 0.003 |
| 30 | 3.569 | 3.224 | 2.913 | 2.699 | 2.816 | 2.898 | 1.603 | 2.168 | 1.284 | 1.179 | 4.133 | 0.003 |
| mean | 3.733 | 3.257 | 2.858 | 2.595 | 2.644 | 2.978 | 1.424 | 2.117 | 1.286 | 1.087 | — | — |
| S.D. | 0.135 | 0.040 | 0.055 | 0.147 | 0.175 | 0.060 | 0.187 | 0.099 | 0.021 | 0.120 | — | — |
| mean-ChrX/ | — | — | — | — | — | — | — | — | — | — | 2.139 | 0.121 |
| Y %-M | | | | | | | | | | | | |
| S.D.-ChrX/ | — | — | — | — | — | — | — | — | — | — | 0.055 | 0.006 |
| Y %-M | | | | | | | | | | | | |
| mean-ChrX/ | — | — | — | — | — | — | — | — | — | — | 4.182 | 0.003 |
| Y %-F | | | | | | | | | | | | |
| S.D.-ChrX/ | — | — | — | — | — | — | — | — | — | — | 0.062 | 0.001 |
| Y %-F | | | | | | | | | | | | |

Furthermore, in order to examine whether the instance of a half chromosome or an additional chromosome existed in the suspected samples, X was assigned to be 50 or 100 and the corresponding chromosomal Z reference value (cutoff value) was calculated (see table 2):

Z=(mean_ChrN %×0.5×X %)/S.D._ChrN %, wherein N represents chromosomes 1-22, X is 50 or 100.

TABLE 2
determination of the reference Z value (cutoff
value) for trisome in the detection cells

| Chromosome | mean_ChrN | S.D._ChrN | Reference Z value | |
|---|---|---|---|---|
| number | % | % | above 50% | above 100% |
| chr1 | 7.9307295 | 0.1159668 | 17.0969881 | 34.1939762 |
| chr2 | 9.0408152 | 0.1458970 | 15.4917815 | 30.9835629 |
| chr3 | 7.3394728 | 0.1633916 | 11.2298783 | 22.4597567 |
| chr4 | 6.8980316 | 0.3000703 | 5.7470125 | 11.4940249 |
| chr5 | 6.6213096 | 0.1717845 | 9.6360687 | 19.2721373 |
| chr6 | 6.3996516 | 0.1482759 | 10.7901077 | 21.5802154 |
| chr7 | 5.4613809 | 0.0819567 | 16.6593481 | 33.3186962 |
| chr8 | 5.2953272 | 0.0912159 | 14.5131676 | 29.0263351 |
| chr9 | 4.0009382 | 0.0572472 | 17.4721938 | 34.9443876 |
| chr10 | 4.8192433 | 0.0693516 | 17.3725054 | 34.7450108 |
| chr11 | 4.6436180 | 0.0780559 | 14.8727280 | 29.7454561 |
| chr12 | 4.6690652 | 0.0647597 | 18.0245772 | 36.0491544 |
| chr13 | 3.7325287 | 0.1346539 | 6.9298575 | 13.8597151 |
| chr14 | 3.2568057 | 0.0398485 | 20.4324240 | 40.8648480 |
| chr15 | 2.8578247 | 0.0553827 | 12.9003599 | 25.8007199 |
| chr16 | 2.5948409 | 0.1465308 | 4.4271266 | 8.8542531 |
| chr17 | 2.6442999 | 0.1753843 | 3.7692929 | 7.5385857 |
| chr18 | 2.9780305 | 0.0598664 | 12.4361514 | 24.8723027 |
| chr19 | 1.4242524 | 0.1868697 | 1.9054083 | 3.8108167 |
| chr20 | 2.1171964 | 0.0985564 | 5.3705200 | 10.7410400 |
| chr21 | 1.2858576 | 0.0205784 | 15.6214179 | 31.2428358 |
| chr22 | 1.0872769 | 0.1204085 | 2.2574758 | 4.5149516 |
| chrX-F | 4.1819271 | 0.0615940 | −16.9737663 | −33.9475326 |
| ChrY-F | 0.0034667 | 0.0006359 | / | / |
| chrX-M | 2.1387665 | 0.0545815 | 9.7962061 | 19.5924123 |
| ChrY-M | 0.1207910 | 0.0055482 | 5.4468013 | 10.8856025 |

The z score_ChrN for each chromosome in the suspected samples was calculated with the following formula:

z score_ChrN=(ChrN % for a given chromosome in the detection samples−mean_ChrN %)/S.D._ChrN %.

TABLE 3
The z score_ChrN for each chromosome in the suspected samples

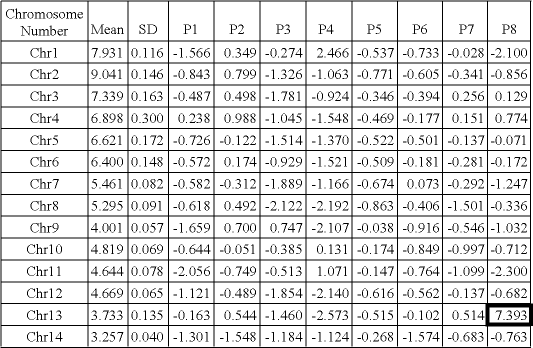

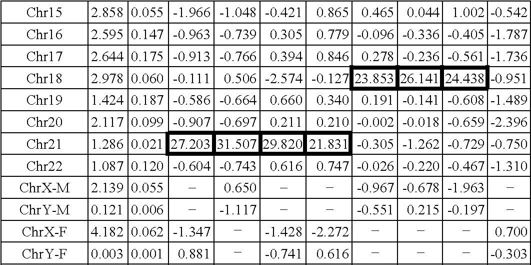

As seen from the analysis above, the suspected samples were 8 in total among the 53 detection samples of amniotic cells, in which, for the chromosomes in each of the suspected samples, 8 abnormalities of chromosome number with the absolute value of a z score_ChrN greater than 3 were detected (see table 3). Specifically, they were:

1) Chr21 for P1, Chr21 for P2, Chr21 for P3, and Chr21 for P4;

2) Chr18 for P5, Chr18 for P6, and Chr18 for P7; and

3) Chr13 for P8.

It was determined by checking the Z value obtained when X=100 in table 2 that the number of chromosome 21 in samples P1-P4 and the number of chromosome 18 in samples P5-P7 were one more than the number of the corresponding chromosomes in the standard cells, respectively, and the number of chromosome 13 in P8 was half one more than the number of the corresponding chromosome in the standard cells. That is, P1-P4 were T21 (Down syndrome), and P5-P7 were 118 (Edwards syndrome), and P8 was mosaic T13 (mosaic Patau syndrome). The results were completely consistent with the traditional analysis results of chromosomal karyotype.

### Example 2

An additional 6 samples (Q1-Q6) of amniotic cells were treated and sequenced in the same way as the above to produce data for analysis. The z score_ChrN was calculated on mean_ChrN % and S.D._ChrN % calculated from 30 standard cellular samples in example 1. 3 positive samples were identified from the 6 samples.

TABLE 4
The ChrN % of 6 detection samples (Q1-Q6)

| | Q1 | Q2 | Q3 | Q4 | Q5 | Q6 |
|---|---|---|---|---|---|---|
| Chr1 % | 7.900099 | 7.781541 | 7.965013 | 7.937310 | 7.835625 | 7.756449 |
| Chr2 % | 9.195581 | 8.969471 | 8.998068 | 9.137041 | 8.836921 | 9.014913 |
| Chr3 % | 7.389485 | 7.365766 | 7.389563 | 7.452117 | 7.134356 | 7.378118 |
| Chr4 % | 7.090694 | 7.005976 | 6.921334 | 7.112517 | 6.510565 | 7.058824 |
| Chr5 % | 6.759707 | 6.600836 | 6.604984 | 6.768853 | 6.357255 | 6.605637 |
| Chr6 % | 6.541994 | 6.468799 | 6.461957 | 6.545516 | 6.170975 | 6.376054 |
| Chr7 % | 5.562187 | 5.423140 | 5.522745 | 5.521768 | 5.342112 | 5.403700 |
| Chr8 % | 5.387074 | 5.344078 | 5.357094 | 5.318220 | 5.176933 | 5.275211 |
| Chr9 % | 3.946516 | 3.924984 | 4.061791 | 4.037918 | 4.007161 | 3.958868 |
| Chr10 % | 4.831699 | 4.680082 | 4.876470 | 4.845395 | 4.798439 | 4.700673 |
| Chr11 % | 4.634992 | 4.541423 | 4.682686 | 4.637077 | 4.603257 | 4.462601 |
| Chr12 % | 4.727456 | 4.552861 | 4.734034 | 4.700509 | 4.571935 | 4.594756 |
| Chr13 % | 3.871131 | 3.749202 | 3.677764 | 3.875716 | 3.475357 | 3.776552 |
| Chr14 % | 3.261377 | 3.247342 | 3.285671 | 3.281681 | 3.238633 | 3.207609 |
| Chr15 % | 2.875226 | 2.782605 | 2.926826 | 2.866104 | 2.830466 | 2.757703 |
| Chr16 % | 2.516559 | 2.443884 | 2.624248 | 2.524383 | 2.665946 | 2.413713 |
| Chr17 % | 2.519897 | 2.481007 | 2.684055 | 2.561488 | 2.725292 | 2.495129 |
| Chr18 % | 3.026389 | 2.939323 | 3.027751 | 2.994205 | 2.893438 | 2.985936 |
| Chr19 % | 1.291162 | 1.240504 | 1.479644 | 1.317938 | 1.482326 | 1.235699 |
| Chr20 % | 2.096249 | 2.063225 | 2.174512 | 2.076472 | 2.173705 | 2.049467 |
| Chr21 % | 1.290966 | 1.267192 | 1.297270 | 1.291611 | 1.896099 | 1.297050 |
| Chr22 % | 0.992960 | 0.989674 | 1.125718 | 1.017386 | 1.164497 | 0.995698 |
| ChrX % | 2.173990 | 4.134076 | 2.117655 | 2.177186 | 4.104752 | 4.197133 |
| ChrY % | 0.116611 | 0.003010 | 0.003148 | 0.001590 | 0.003956 | 0.002508 |

TABLE 5
The z score_ChrN for 6 samples calculated from the mean-ChrN% and
S.D._ChrN% of 30 negative samples in example 1

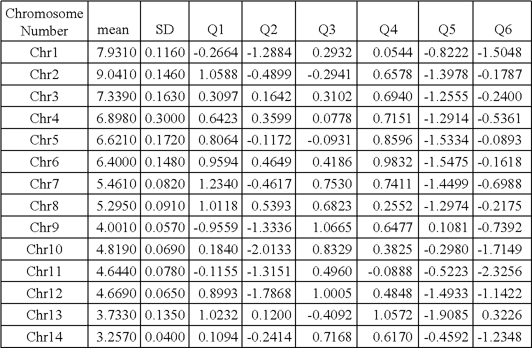

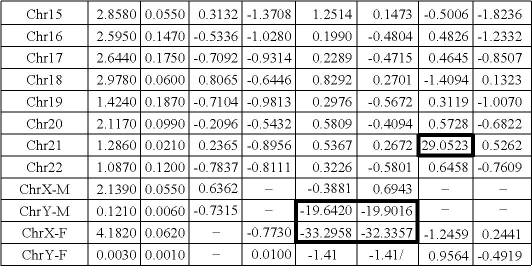

As seen from the results, Q5 had an extra copy of chromosome 21 than the standard cells, which was T21; Q3, Q4 missed one copy of chromosome X, which was 45×0 (Turner syndrome). The results were completely consistent with the traditional analysis results of chromosomal karyotype.

Although the examples of the invention have been described in great detail, a person skilled in the art will understand that, according to all of disclosed teachings, a variety of modification and replacement may be made of those details. The changes are covered by the scope of protection of the invention. The whole scope of the invention is defined by attached claims and its equivalent.

1. A method of using sequencing to analyze the chromosomal information of cells, including the steps of:
a. randomly breaking a genome DNA of the cells to obtain DNA fragments of a certain size, and sequencing them;
b. strictly aligning the DNA sequences sequenced in step a with a reference sequence of the human genome to obtain information about the DNA sequences being located on a particular chromosome;
c. for a particular chromosome N, determining a total number of the sequences, located at a sole region of the chromosome, among the above-sequenced DNA sequences, thereby calculating an ChrN % for chromosome N, i.e. a ratio of a total number (S1) of the sequences located at the sole region of chromosome N, among the above-sequenced DNA sequences, to a total number (S2) of the sequences located on all chromosomes, among the above-sequenced DNA sequences:
ChrN %=the total number of the sequences located at the sole region of chromosome N/the total number of the sequences located on all chromosomes; and
d. comparing the ChrN % for chromosome N with a ChrN % for the corresponding chromosome from standard cells to determine whether there exists a difference between the chromosome of the cells and a chromosome of a corresponding standard cell.

2. The method of claim 1, wherein strictly aligning with the reference sequence of the human genome described in step b means that the method of alignment adopted is a fault-intolerant alignment of the sole region located in the reference sequence of the human genome; wherein the reference sequence of the human genome is produced after a shield of the repeated sequences within the human genome sequence.

3. The method of claim 1, wherein determining whether there exists a difference between the number of the particular chromosome in the cellular samples and the standard cells in step d is accomplished by drawing a boxplot, wherein out of the samples, a sample for which the ChrN % corresponds to an outlier that goes beyond 1.5-3 times or above 3 times an interquartile range, wherein the outlier is determined to differ from the standard cells in the chromosome number, i.e. aneuploidy.

4. The method of claim 1, wherein determining whether there exists a difference between the number of a particular chromosome in the cellular samples and in the standard cellular samples in step d is accomplished by using a “z score_ChrN” to indicate the deviation of the ChrN % for the said cellular samples from the ChrN % for the standard cellular samples, if an absolute value of the z score_ChrN is greater than or equal to 3, there exists a difference in the number of the particular chromosome between the cellular samples and the standard cells.

5. The method of claim 4, wherein:
the z score_ChrN=(the ChrN % for the particular chromosome from the detection samples—a ChrN % mean (mean_ChrN %) for the particular chromosome)/a ChrN % standard deviation (S.D._ChrN %);
wherein the mean_ChrN % mean for the particular chromosome may be determined according to the ChrN % for the chromosome from at least 10, preferably at least 20, standard cellular samples; and
wherein the S.D._ChrN % mean for the particular chromosome may be determined according to the average value of mean_ChrN % for the chromosome from at least 10, preferably at least 20, standard cellular samples.

6. The method of claim 4, wherein determining whether there exists a difference between the number of the particular chromosome in the cellular samples and in the standard cellular samples in step d is accomplished by comparing the z score_ChrN with a Z reference value, wherein the Z reference value is determined by the following method:
Z=(mean_ChrN %×0.5×X %)/S.D._ChrN %
Wherein X may be any integer between, inclusive, negative 100 (i.e. −100) and positive 100, for example −100, −90, −80, −70, −60, −50, −40, −30, −20, −10, 0, 10, 20, 30, 40, 50, 60, 70, 80, 90, 100;
When the absolute value of the z score_ChrN is greater than or equal to 3 and reaches the absolute value of the Z reference value, there is an X % difference between the number of the particular chromosome in the cells and that in the standard cells.

7. The method of claim 1, wherein the cells are amniotic cells, such as uncultured amniotic cells or cultured amniotic cells, and preferably uncultured amniotic cells.
